(19) United States
(12) Patent Application Publication
(10) Pub. No.: US 2010/0112044 A1
(43) Pub. Date: May 6, 2010

(54) METHOD AND APPARATUS FOR ENCAPSULATING PHARMACEUTICAL AND NUTRICEUTICAL BIOACTIVES FOR INTESTINAL DELIVERY

(76) Inventor: Daniel R. DeBrouse, Oklahoma City, OK (US)

Correspondence Address: DUNLAP CODDING, P.C., PO BOX 16370, OKLAHOMA CITY, OK, 73113, US

(21) Appl. No.: 12/648,901

(22) Filed: Dec. 29, 2009

Related U.S. Application Data
(63) Continuation of application No. 11/166,385, filed on Jun. 24, 2005, now abandoned.
(60) Provisional application No. 60/582,632, filed on Jun. 24, 2004.
(60) Provisional application No. 60/582,633, filed on Jun. 24, 2004.

(51) Int. Cl. A61K 9/14; A61K 47/26; A61K 38/00; A61K 31/7088; A61K 48/00; A61K 38/28; A61K 9/48

(52) U.S. Cl. 424/451; 514/777; 514/2; 514/44 A; 514/44 R; 514/3; 424/489

(57) ABSTRACT

A method for the encapsulation and subsequent delivery of a biologically, water or lipid soluble, active agent to the human intestinal mucosa. This biochemical pathway to drug delivery includes the steps of forming an aqueous emulsion of the pharmacological or nutriceutical agent, vegetable oil, gum and/or gum resin, absorbent factors, and a sugar; then converting the emulsion into a dry spherical particulate form, having lost its aqueous component; and then drying the resultant particulate to form acid and water insoluble intestinal absorbent biologically active beadlets.

### CROSS REFERENCE TO RELATED APPLICATIONS

The present application is a continuation of U.S. Ser. No. 11/166,385, filed Jun. 24, 2005, which claims the benefit under 35 U.S.C. 119(e) of U.S. Provisional Patent Application Ser. No. 60/582,632, filed Jun. 24, 2004, and U.S. Provisional Patent Application Ser. No. 60/582,633, filed Jun. 24, 2004, each of which is explicitly incorporated herein by reference in its entirety.

### STATEMENT REGARDING FEDERALLY SPONSORED RESEARCH OR DEVELOPMENT

Not applicable.

### TECHNICAL FIELD

The present invention relates generally to pharmaceutical and nutriceutical products and more particularly to an improved method and apparatus for encapsulating pharmaceutical and nutriceutical bioactives for intestinal delivery.

### BRIEF SUMMARY OF THE INVENTION

The method and apparatus for encapsulating pharmaceutical and nutriceutical bioactives for intestinal delivery of the present invention provides a vehicle for oral administration of a protected biologically active agent for subsequent delivery to the small intestine of a mammal. The vehicle bestows protection by encapsulating a bioactive within an inner core region, preventing its disintegration and thus dissolution, until the spherical particulate reaches the juices of the small intestine. Furthermore, the bioactive is protected from enzymatic degradation through the formula until absorption at the intestinal mucosa. In addition, the formulation greatly enhances the bioavailability of bioactives through a provision of absorbent factors, specifically targeting intestinal mucosa receptors.

It is therefore an object of the present invention to provide a new and improved method for encapsulating pharmaceutical and nutriceutical bioactives.

It is another object of the present invention to provide a new and improved vehicle for oral administration of a protected biologically active agent.

A further object or feature of the present invention is a new and improved encapsulation of a bioactive to prevent premature disintegration, dissolution, and enzymatic degradation.

An even further object of the present invention is to provide a novel method to enhance the bioavailability of bioactives through a provision of absorbent factors, specifically targeting intestinal mucosa receptors.

Other novel features which are characteristic of the invention, as to organization and method of operation, together with further objects and advantages thereof will be better understood from the following description, in which preferred embodiments of the invention are illustrated by way of example. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### DETAILED DESCRIPTION OF THE INVENTION

This invention relates to the formulation and production of water insoluble pharmacologically or nutriceutically active beadlet for oral delivery to the small intestine of a mammal. The invention includes the preparation of an emulsion containing the bioactive(s) material, vegetable oil, gum and gum resins, absorbent factors, and sugar(s); maintaining the emulsion at a temperature between −20 and 200 degrees Celsius for droplet conversion; the collection of warm or cooled emulsion droplets individually in a mass of collectant powder or salt water such as calcium chloride, which are separated in space from one another until their morphological particulate form has been established; and the separation of bioactive beadlet particles from the collectant powder followed by drying to 0-15 percent final moisture content.

Pharmacological active agents which may be used in the practice of this invention include but are limited to antibiotics such as cephalosporin, antiviral agents such as interferon or protease inhibitor, anti-inflammatory agents, anti-tumor agents, chemotherapeutic agents, polypeptides, steroidal agents, anti-sense agents, RNA agents and DNA agents, insulin, and immunosuppressants. Nutriceutical active components which may be used include but are not limited to caroteniods, vitamins, minerals, phototropic agents and anthrocyanins. The bioactive agent preferably comprises 0-50 percent of the composition by weight.

The first process step in the practice of this invention comprises emulsifying the bioactive component in one or more types of oil bases, a combination of gums and gum resin, absorbent factors and one or more sugars.

Any combination of pharmaceutical grade oil such as the following may be suitable for this process; soybean oil, sesame oil, safflower oil, canola oil, cotton seed oil, olive oil, corn oil, sunflower oil and or vegetable oil. The oil preferably comprises 1 to 80 percent of the composition by weight.

Among the suitable gums and gum resins which may be used in combination include, cellulose gum, pectin and its resins, locust bean gum, resins and derivatives, xanthan gum and resins, carrageenan and derivatives, sodium salt of carrageenan, gellan gum and resins, whey protein gum and resins, agar agar, propylene glycol alginate, derivatives and resins, Arabic gum and resins, guar gum and resins, gum traqacanth, and gum ghatti. The gum and gum resin preferably comprise 0.5-10 percent of the composition by weight.

Suitable absorbent factors include but are not limited to glycyrrhizinate, glycrrhetinic acid, sucrose fatty acid ester, glycerin, glycerol fatty acid ester, adipic acid, polyethylene glycol, sodium dodecyl sulfate, sodium caprate, and sodium deoxycholate, and any combination thereof. The absorbent factor preferably comprises 0.5-30 percent of the composition by weight.

Suitable sugars include, mannose, dextrose, fructose, maltose, sucrose, glucose, lactose and their derivatives and combinations thereof. Sugars are most typically added in combination but may be added alone when appropriate. The sugar preferably comprises 0.5-20 percent of the composition by weight.

The emulsion of this invention may also contain small quantities of butylated hydroxy toluene, glycerin, polyethylene glycols, propylene glycol, lecithin, antioxidants, tocopherol, docosahexaenoic acid, and pirotiodecane in addition to coloring agents, solubilizers and extenders.

The emulsion of this invention may be prepared and maintained, collected and dried by methods traditional to those skilled in the art. As an example, the following will outline a very general satisfactory method, with the understanding that several other practices apply as well. For example, the beadlet solution may be mixed in some cooled state and atomized and collected in a salt water solution directly. A combination of appropriate gums are diluted with 5-30 times their combined weight amount in distilled deionized water and heated to their combined melting point, or cooled in cold gelation. The biologically active material is suspended in a combination of oils and solubilizers and added to a mixture of absorbent factors, sugars, colorants and any other necessary components as previously outlined. Once the aqueous gum solution has reached its melting or cooling point the temperature is lowered or held to a range between 30 and 200 degrees

Celsius and held at such a temperature. At this time the bioactive composition is added and the emulsion is homogenized. The emulsion temperature is again adjusted to a holding range of −20 to 200 degrees Celsius for droplet conversion.

The powder used for the collection of emulsion droplets may consist entirely of flour, starch or chemically modified starches in addition to a number of other suitable collectants. The collectant powder may also contain quantities of other components to increase its effectiveness. They preferably meet the following criteria to be suitable in practice; it should possess an initial moisture content of less than 15 percent, it should be relatively insoluble in cold water, it should be resistant to water wetting, and it should possess a high capacity to absorb water. In addition as mentioned previously, a salt water solution may be used as collectant as well. The only requirement being that the salt used preferably be calcium or potassium in nature.

Various methods exist which may be used in the practice of this invention to introduce the bioactive emulsion droplets into the collectant powder or water. The only requirement is that one chooses a procedure which is effective in maintaining space separation between the individually collected droplets. For example, bioactive emulsion droplets may be atomized through a stationary or moving nozzle upon a stationary or moving or rotating layer of collectant powder, or water bed reservoir. In a further example the bioactive emulsion droplets may be sprayed or gravity dropped into an agitated cloud of collectant powder.

After a set-up period of 15 minutes to 24 hours the emulsion droplets have been morphologically established into a spherical particulate form. At this point the established bioactive beadlets ranging in size from 40-250 mesh may be separated from the powder collectant by any of a number of traditional practices. For example, an appropriate sized shaking screen may be employed to gravity separate collectant powder from the bioactive beadlets. A more rapid and automated procedure would involve the use of commercial automatic multiple deck separators, such as are common within the industry.

Once separated from collectant, the bioactive beadlets are dried by conventional methods preferably within the range of −20 to 200 degrees Celsius for a period of 30 seconds to 20 minutes, to a final moisture content ranging from 0-15 percent moisture by weight.

The final bioactive beadlet particles may then be compressed or filled in tablet and capsule formulations for the oral administration to mammals.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. A method for the production of a bioactive agent delivery system, said method comprising the steps of:
forming an aqueous emulsion of the bioactive agent, vegetable oil, gum and/or gum resin, absorbent factors, and sugar(s); and
converting the emulsion into a dry spherical particulate form.

2. The method of claim 1 further including the step of:
heating the spherical particulate form within the range of −20 to 200 degrees Celsius for a period of 30 seconds to 20 minutes, having a final moisture content of 0 to 15 percent.

3. The method of claim 1 wherein the bioactive agent is selected from the group consisting of an antibiotics, antiviral agents, anti-inflammatory agents, anti-tumor agents, chemotherapeutic agents, polypeptides, steroidal agents, anti-sense agents, RNA agents and DNA agents, insulin, and immunosuppressants.

4. The method of claim 1 wherein the bioactive agent is selected from the group consisting of caroteniods, vitamins, minerals, phototropic agents and anthrocyanins.

5. The method of claim 1 wherein the bioactive agent comprises 0-50 percent of the composition by weight.

6. The method of claim 1 wherein oil comprises 1 to 80 percent of the composition by weight.

7. The method of claim 1 wherein the gum and gum resin comprise 0.5-10 percent of the composition by weight.

8. The method of claim 1 wherein the absorbent factor comprises 0.5-30 percent of the composition by weight.

9. The method of claim 1 wherein the sugar comprises 0.5-20 percent of the composition by weight.

10. The method of claim 1 wherein the dry spherical particulate ranges in the size of 40-250 mesh.

11. The method of claim 1 wherein the dry spherical particulate is packaged into an oral capsule for administration to a patient.

12. A water insoluble pharmacologically or nutriceutically active beadlet for oral delivery to the small intestine of a mammal, said beadlet comprising:
a spherical particulate form having a moisture content of 0 to 15 percent;
said spherical particulate comprising a bioactive agent selected from the group consisting of an antibiotics, antiviral agents, anti-inflammatory agents, anti-tumor agents, chemotherapeutic agents, polypeptides, steroidal agents, anti-sense agents, RNA agents and DNA agents, insulin, immunosuppressants, caroteniods, vitamins, minerals, phototropic agents and anthrocyanins, said bioactive agent comprising 0-50 percent of the composition by weight; oil comprising 1 to 80 percent of the composition by weight;
gum and gum resin comprising 0.5-10 percent of the composition by weight;
absorbent factor comprising 0.5-30 percent of the composition by weight; and
sugar comprising 0.5-20 percent of the composition by weight.

13. The beadlet of claim 12 wherein the spherical particulate ranges in the size of 40-250 mesh.

14. The beadlet of claim 12 wherein a plurality of beadlets are filled capsule formulation for the oral administration to mammals.
